(19) United States
(12) Patent Application Publication
(10) Pub. No.: US 2010/0081875 A1
(43) Pub. Date: Apr. 1, 2010

(54) Surgical Device For Minimal Access Surgery

(75) Inventors: Dennis Fowler, Boston, MA (US); Peter Allen, Pleasantville, NY (US)

Correspondence Address: SILLS CUMMIS & GROSS P.C., ONE ROCKEFELLER PLAZA, IP DEPARTMENT - 25TH FLOOR, NEW YORK, NY, 10020, US

(73) Assignee: EndoRobotics Inc., New York, NY (US)

(21) Appl. No.: 12/545,403

(22) Filed: Aug. 21, 2009

Related U.S. Application Data
(63) Continuation-in-part of application No. 11/475,737, filed on Jun. 26, 2006.
Application No. 11/475,737 is a continuation of application No. 11/226,665, filed on Sep. 13, 2005, now abandoned.
Application No. 11/226,665 is a continuation-in-part of application No. 10/620,298, filed on Jul. 15, 2003, now Pat. No. 7,066,879.
(60) Provisional application No. 61/218,316, filed on Jun. 18, 2009.

(51) Int. Cl. A61B 17/34; A61B 1/00

(52) U.S. Cl. 600/114; 604/164.01

(57) ABSTRACT

The present invention is directed to devices and methods for performing minimal access surgery; in various embodiments, to a surgical device comprising an insertable instrument attached to a trocar, as well as methods of performing a surgical procedure comprising inserting in insertable instrument/trocar combination into the body of a patient through an incision.

### CROSS REFERENCE TO RELATED APPLICATIONS

The present application claims benefit of provisional Application No. 61/218,316 filed Jun. 18, 2009. This application is also a continuation-in-part of application Ser. No. 11/475,737, filed on Jun. 26, 2006, which is a continuation of application Ser. No. 11/226,665, filed on Sep. 13, 2005, now abandoned, which is a continuation-in-part of application Ser. No. 10/620,298, filed on Jul. 15, 2003, now U.S. Pat. No. 7,066,879; all of which are hereby incorporated by reference in their entireties.

### FIELD OF THE INVENTION

This invention relates to the field of devices, in particular, devices that are useful in procedures such as minimal access or minimally invasive surgery, and that can be inserted and temporarily placed or implanted into a structure having a lumen or hollow space, such as a patient's abdominal cavity or elsewhere within the body of a patient, to provide access to a site of interest in connection with such minimal access or minimally invasive surgery.

### BACKGROUND OF THE INVENTION

Minimal access surgery, also known as minimally invasive surgery, often requires the use of multiple incisions on a patient's body for insertion of devices therein. Generally, in this type of surgery, small incisions (usually only millimeters long) are made in the surface of a patient's body, permitting the introduction of probes, scopes and other instruments into the body cavity of the patient. In this way, a number of surgical procedures can be performed without the need for large surgical incisions, thus minimizing recovery time.

In these types of internal procedures, one incision is generally used for the insertion of a scope into a patient's body, and additional incisions are used for the insertion of additional surgical tools. Some procedures in the art further involve the insertion of a trocar (which may also be known as a port or muscle tissue dilator) through the skin of a patient to keep the incision open for ease of access to the patient's interior during the procedure.

For most procedures involving either the abdominal or chest cavity, the surgeon will use two or more additional small incisions through which various surgical instruments are inserted, such as a device containing a camera that allows monitoring of the procedure at a remote location, or additional instruments that provide means for removal of bodily fluids during the procedure, or to dissipate pressure on or near the location of the procedure. The multiple incisions have been found to be overall less invasive, and require a shorter recovery time, than a single large incision.

However, even though multiple incisions are small, each is still often a source of pain for a patient, each may still result in scarring, and each still has a small but finite likelihood of leading to complications such as a wound infection or incisional hernia. The greater the number of incisions, the incrementally greater likelihood of complications.

In the past, reducing the number of incisions has been attempted rarely, and with limited success. Abdominal surgery through a single incision has been attempted. Generally, in such procedures the surgeon makes an incision that is large enough to accommodate the insertion of a single trocar, where the trocar must be large enough to insert both a scope and multiple additional instruments necessary for the surgery. However, these procedures require a different level of skill on the part of the surgeon, as well as different instruments than typically used when multiple incisions are made.

Another method involves making a larger incision (generally 1.5 to 2.0 cm at the umbilicus) through which multiple trocars having a diameter of 3, 4 or 5 mm each can be inserted through the same skin incision. However, the surgeon must still use a laparoscope, and the laparoscope generally either occupies space in the large port that could otherwise be used for instruments for the operation, or the laparoscope requires its own port placed through the larger skin incision. In each case, the laparoscope has the tendency to get in the way of the other instruments or otherwise complicate the surgery, further diminishing the surgeon's ability to manipulate the instruments and visualize the site of the surgery.

Additionally, each trocar requires a separate fascial incision that is, a separate incision the layer of tissue below the skin of the patient. This is true even though after the procedure is finished, it appears that only one incision was made because there is only a single incision in the skin itself. Thus, in all of the previously known methods in the art, it remains desirable to reduce the diameter of the incision required.

Reducing the number of incisions statistically lessens the likelihood of infection in a procedure. Therefore, it is desirable to provide a surgical device that permits an operator to maximize the amount of information that can be obtained through a procedure and maintain the ease of performing the procedure with few complications, while minimizing the likelihood of infection, discomfort and complications to a patient. It is further desirable to provide systems and devices for minimal access procedures that do not require an assistant to hold and orient an instrument, and that provide additional or greater freedom with regard to orienting the instrument toward a site of interest.

### SUMMARY OF THE INVENTION

In certain embodiments, the present invention is directed to a surgical device that can be used in minimal access surgery. The surgical device may comprise an insertable instrument attached to a trocar to provide an insertable instrument/trocar combination. The surgical device may be capable of being inserted into the body of a patient via a small incision, to permit operation on the patient. The insertable instrument may comprise an imaging device.

The present invention also provides a system and single or multi-functional element insertable instrument that can be inserted and temporarily placed or implanted into a structure having a lumen or hollow space, such as a subject's abdominal cavity to provide access to the site of interest in connection with minimally invasive surgical procedures. The insertable instrument may be configured such that the functional elements have various degrees of freedom of movement with respect to orienting the functional elements or elements to provide access to the site from multiple and different orientations/perspectives as the procedure dictates, e.g., to provide multiple selectable views of the site, and may provide a stereoscopic view of the site of interest.

In certain embodiments, the present invention is directed to a surgical device comprising:

(a) an insertable instrument; and

(b) a trocar attached to the insertable instrument.

In other embodiments, the present invention is directed to a surgical device comprising an insertable instrument attached to a trocar to provide an insertable instrument/trocar combination,

wherein the insertable instrument is capable of being inserted into a single incision in the skin of a patient to permit a surgical procedure on the patient.

In certain embodiments, the present invention is directed to method of performing a procedure on a patient, the method comprising the steps of:

(a) making an incision in the skin of a patient to provide an opening having a diameter of about 3 cm or less; and

(b) inserting an insertable instrument having a trocar attached thereto through the opening.

In other embodiments, the present invention is directed to method of performing a surgical procedure on a patient, the method comprising:

(a) inserting a trocar into an incision on the surface of a patient's body, such that the trocar touches the incision and maintains an opening in the skin of the patient;

(b) inserting an insertable instrument into the interior of a patient's body through the trocar; and

(c) attaching the trocar to the insertable instrument via a point of attachment on the surface of the trocar or the insertable instrument; wherein at least a portion of the point of attachment is within the interior of the patient's body.

In other embodiments, the present invention is directed to method of performing a surgical procedure on a patient, the method comprising:

(a) making an incision in the skin of a patient to provide an opening having a diameter of about 3 cm or less;

(b) attaching a trocar to an insertable instrument to form an insertable instrument/trocar combination; and

(c) inserting at least a portion of the insertable instrument/trocar combination through the incision into the patient's body, such that the trocar touches the incision and maintains an opening in the skin of the patient, and such that at least a portion of the insertable instrument is within the interior of the patient's body.

In various embodiments of the present invention, the methods are directed to the additional step of inserting one or more additional items through the opening, the one or more additional items chosen from any additional insertable instrument as defined herein, or any other available laparoscopic or minimally invasive surgery tool useful for the procedure.

In various embodiments, the present invention is directed to the additional steps of performing a procedure on the patient such as, for example, surgery, an exploratory procedure, a procedure with or without biopsy or other technique to obtain a specimen, treatment of an interior cavity or surface of a patient, removal of tissue or organ material from a patient, or delivery of therapeutic agents (such as, e.g., chemotherapy, seeds or other vehicles for delivering radiotherapy and the like).

### BRIEF DESCRIPTION OF THE DRAWINGS

FIG. 1 shows a device known in the art, specifically, the pNavel® device for single incision surgery. This device includes a port for a laparoscope, but does not teach that the port can be attached to an insertable instrument.

FIG. 2 shows an insertable instrument/trocar combination in accordance with certain embodiments of the present invention, specifically, a laparosurgical instrument/trocar combination. Depicted therein are: one or more orifices for instruments, and external end of the port used for insufflation, the camera module attached to the insertion end of the trocar.

FIG. 3 shows an insertable instrument/trocar in accordance with another embodiment of the present invention, wherein the insertable instrument is an endoscope that further comprises an actuator (in this case, a knob) for mechanical control of the camera pan and tilt freedoms; as well as one or more cables or pulleys for control, and one or more carbon dioxide (CO₂) ports.

FIG. 4 shows a surgical device in accordance with another embodiment of the present invention, wherein the surgical device comprises a camera controlled through motorized computer control. The surgical device comprises an endosurgical instrument attached to a trocar and comprising a camera and one or more cables of wires, one or more motors, and one or more gears (screwdrives) for control of the camera; as well as a worm gear.

FIG. 5 shows a view of camera module in accordance with one embodiment of the present invention.

FIG. 6 shows an alternate side view of a camera module in accordance with another embodiment of the present invention.

FIG. 7 shows a side view of an insertable instrument in accordance with another embodiment, separate from any attachment to a trocar. Pictured are the camera module attached to the end of the trocar, and a mechanism for pan and tilt movement.

FIGS. 8(a) and 8(b) show two views of an insertable instrument/trocar combination according to certain embodiments the present invention, comprising two cameras, each exhibiting 5 degrees of freedom. FIG. 8(a) shows an insertable instrument/trocar combination with the cameras retracted, and FIG. 8(b) shows the insertable instrument/trocar combination with the cameras extracted.

FIG. 9 shows another embodiment of the present invention, wherein the insertable instrument is an in vivo imaging device having a single camera, but wherein the platform can be adapted to contain two cameras.

FIG. 10 shows another embodiment of an insertable instrument according to the present invention, from a top view and a side view, as described in Example III.

FIG. 11 shows an insertable instrument according to the present invention, as described in Example III, highlighting the LED, CCD, pan and tilt capabilities, the motor and the worm.

FIG. 12 shows the system configuration in accordance with the surgical devices of certain embodiments of the present invention.

### DETAILED DESCRIPTION OF THE INVENTION

Throughout the disclosure of the present invention, the disclosures of all references cited are hereby incorporated by reference in their entirety. In the case of any conflicts in definitions between the disclosures of such references and the present disclosure, the present disclosure controls.

Throughout the disclosure of the present invention, the term “surgical device” means any device comprising mechanical and/or electrical parts that is useful for surgery on a patient.

Throughout the disclosure of the present invention, “inserted” refers to at least partial insertion of an object, or insertion of any a portion of any object in other words, an object need not be entirely disposed within a patient's body to qualify as “inserted.” Specifically, in certain and non-limiting embodiments,

1. the trocar may be entirely inserted within the patient's body, but the insertable instrument attached thereto may be only partially inserted;

2. the insertable instrument may be entirely inserted within the patient's body, but the trocar attached thereto may only be partially inserted; or may actually not be inserted at all, e.g., at least a portion of the trocar may lie on the surface of the patient's body, keeping the incision open to ease insertion of the insertable instrument; or

3. both the trocar and the insertable instrument may be partially or entirely inserted within the patient's body.

In various embodiments, any portion or element of the surgical devices of the present invention (including the trocar and any part of the insertable instrument) may be rigid or flexible, and may be made of from a variety and/or a combination of biocompatible and non-biocompatible materials, such as polyester, Gortex, polytetrafluoroethyline (PTFE), polyethylene, polypropylene, polyurethane, silicon, steel, stainless steel, titanium, Nitinol, or other shape memory alloys, copper, silver, gold, platinum, Kevlar fiber, carbon fiber, plastic or other polymeric material, fiberglass, glass, rubber or any other material that is inert, non-reactive, not harmful to the patient and capable of being sterilized in connection with the surgical procedure. Where non-biocompatible materials may come into contact with anatomic structure, the components made from the non-biocompatible materials may be covered or coated with a biocompatible material. In one embodiment, a housing of the insertable instrument is manufactured from stainless steel. The one or more housings may be stainless steel tubes of various diameters.

Insertable Instrument As used herein, “insertable instrument” means any instrument that may be inserted into the interior of a patient for a procedure, including an exploratory or surgical procedure, that comprises a housing, and that is capable of being attached to a trocar to provide a surgical device according to the present invention.

In certain embodiments, the present invention provides a single or multi-functional element insertable instrument that can be inserted and temporarily placed or implanted into a structure having a lumen or hollow space. The structure having the lumen may be the anatomical structure of a subject, such as a patient's heart, lungs, esophagus, stomach, intestines, thoracic cavity, abdominal cavity, blood vessels, ducts, vagina, bladder, etc. and non-anatomical structure, such as tanks, pipes, confined spaces, rooms, etc. In one embodiment, the present invention is adapted to be inserted and temporarily implanted into a patient's abdominal cavity to provide images of a site for use in connection with minimally invasive surgical procedures. The patient may be a human or any mammal.

Once inserted into the lumen of the structure, the insertable instrument may be removably attached or secured to the interior of the structure, such as to the interior of a patient's abdominal wall, near a site of interest so that the functional element or elements may be oriented to the area of interest. The insertable and implantable aspect of the present invention obviates the limited motion about an insertion point drawback associated with endoscopes, as well as other instruments known in the art, by allowing the surgeon to, in various embodiments, move the insertable instrument to different locations on the abdominal wall. Moreover, the insertable aspect allows a surgeon to insert a plurality of insertable instruments into the structure's lumen through a single incision, thereby increasing access to the site with minimal incisions.

Although the present invention may be described by way of example in relation to minimal invasive surgical procedures, it is understood that the invention is equally applicable to provide images, as well as various other functionalities, of numerous structures with a lumen, and is therefore not limited thereto. Imaging is used herein to generally denote pertaining to producing an image of a site, such as producing a video image of a surgical site.

In certain embodiments, the insertable instrument may comprise any instrument known in the art, so long as it comprises a housing or is connected to a housing. For example, an insertable instrument according to the present invention may comprise a scope, a sharp edge, a cutting implement (such as a scalpel or a razor), a dissector, a retractor, a cauterizer, a suction device such as, e.g., a suction hose, a hydroaspirator or the like; a probe, a magnifier, scissors, tweezers, forceps, a coagulator, an ultrasonically activated shears or probe, or other monopolar or bipolar cutting and coagulating devices, or any other energy source for cutting, dissection, or hemostasis; as well as a delivery device for an adhesive, anti-adhesive or hemostatic agent, a mesh, a prosthesis or an implantable device; or any surgical device comprising a housing having a generally tubular or elongated portion, to assist in ease of insertion into a small incision.

In some embodiments, the insertable instrument is a scope. As used herein, the term “scope” means any general scope or any of many sub-categories of specialized scopes that can be used for insertion into, or aid in procedures directed to, any part of a patient's body for example, but not limited to the following: any interior of a patient's body (endoscope), the abdominal or pelvic cavity (laparoscope), the chest cavity (thoracoscope), the colon (colonoscope), the gastric cavity (gastroscope), the interior of a joint (arthroscope), the bladder (cystoscope), esophagus, stomach and duodenum (laryngoscope, esophoagoscope, esophagogastroduodenoscope), the bile duct (endoscopic retrograde cholangiopancreatography (ERCP), duodenoscope-assisted cholangiopancreatoscopy, intraoperative cholangioscopy), the nose (rhinoscope), the lower respiratory tract (bronchoscope), the ear (otoscope), the urinary tract (cystoscope), the cervix (colposcope), the uterus (hysteroscope), the Fallopian tubes (falloscope), the organs of the chest (thoracoscope or mediastinoscope), the amnion of a pregnant patient (amnioscope), a fetus (fetoscopy), in plastic surgery applications (panendoscope, laryngoscope), and the like.

In various embodiments, the scope may be rigid or flexible, and may provide direct image transmission or fiberoptic image transmission, or may be, e.g., a videoscope with a video camera in it.

In certain embodiments, the insertable instrument may have one or more functional elements configured to have or exhibit various degrees of freedom of movement with respect to orienting the functional elements. As used herein, “functional element” means an instrument or device that provides a desired functionality with regard to the minimal access procedure; e.g., a data acquisition device, such as a camera element, a sensor, an ultrasound probe, or an effector, such as a light element, a laser element, a grasper, a dissecting instrument, a needle, a scalpel, a grasper, dithermy/cautery instruments, a suturing instrument, a stapling instrument, or any other insertable instrument discussed in the present disclosure.

Where the functional element or element is a camera element, the insertable instrument may provide a wider field of view of the surgical site than that provided by standard endoscopic cameras. Additionally, the insertable instrument may be so configured to provide access to a site of interest from multiple and different orientations or perspectives within the lumen, as the procedure dictates, further obviating limited mobility about the point of insertion drawback associated with endoscopes. In a multi-camera element embodiment of the invention, the imaging device provides multiple selectable views of the site and may be used in connection with a stereoscopic imaging system to provide a stereo view of the surgical site to recreate the sense of depth that is lost with a traditional video monitor.

In certain embodiments, the insertable instrument comprises a first housing, at least one functional element connected to the first housing, the functional element for use during a minimal access procedure, and a securing element for removably securing the insertable instrument to or against a wall of a structure having a lumen. In one embodiment, the at least one functional element is movably connected to the first housing, and the insertable instrument further includes at least one actuating element connected to the first housing and the functional element. The actuating element is generally capable of moving the functional element in relation to the first housing in at least one degree of freedom. The securing element may be a needle protruding from the imaging device essentially inline with the elongated axis of the device, such as a magnet, a clamp or an adhesive. The insertable instrument may be adapted or otherwise configured for use in connection with minimal access surgical procedures. In this instance, the securing element may include a needle protruding from the insertable instrument essentially inline with the elongated axis of the insertable instrument, enabling the insertable instrument to be removably secured against the wall of a patient's interior by inserting the needle into tissue of the wall.

In one embodiment, at least one functional element is movably connected to the first housing and the insertable instrument includes at least one actuating element connected to the first housing and the functional element. In this instance, the actuating element is capable of moving the camera element in relation to the first housing in at least one degree of freedom selected from a group consisting of: a first degree of rotational freedom essentially orthogonal to the elongated axis; a second degree of rotational freedom essentially inline with the elongated axis; and a third degree of translation freedom essentially inline with the elongated axis.

In certain embodiments, the at least one functional element is a plurality of camera elements movably connected to the first housing, and the insertable instrument includes a plurality of actuating elements connected to the first housing and the camera elements. In this instance, the actuating elements are capable of moving each of the camera elements in relation to the first housing in at least one degree of freedom selected from the group noted above.

In another embodiment, the at least one functional element is movably connected to the first housing and the insertable instrument includes at least one actuating element connected to the first housing and the functional element. In this instance, the actuating element is capable of moving the functional element in relation to the first housing in a third degree of longitudinal freedom essentially inline to the elongated axis allowing the functional element to translate along the third degree of freedom.

Movement in a third degree of longitudinal freedom may be accomplished with a functional element that is mounted to a shuttle capable of moving along the elongated axis. The actuating element may be a motor producing rotational movement connected to a lead screw that interfaces with a threaded portion of the shuttle to translate the rotational movement of the motor into longitudinal movement in the shuttle along the elongated axis. Such movement may also be accomplished for a plurality of functional elements with a corresponding number of motors producing rotational movement, and a corresponding number of shuttles each functional element is mounted to a shuttle capable of moving along the elongated axis. In this instance, each shuttle includes a threaded portion and a hole, and each motor connected to a lead screw interfaces with the threaded portion of one of the shuttles to translate the rotational movement of the motor into longitudinal movement in the shuttle along the elongated axis, and each lead screw passes through the hole of another shuttle to provide a guide for the other shuttle. Each shuttle may include mounted thereto at least one actuating element capable of moving the functional elements in relation to the first housing in a first degree of rotational freedom essentially orthogonal to the elongated axis allowing the functional elements to be retracted into and extracted from the first housing. The plurality of actuating elements may be capable of moving each of the functional elements independently of each other. The translational movement may also be accomplished with a linear rail/actuator system.

In other embodiments, the insertable instrument includes a second housing rotatably attached to the first housing and at least one actuating element connected to the first and second housings. In this instance, the actuating element is capable of rotating the first housing in relation to the second housing and each housing has an access opening therein capable of aligning with each other so that the first housing may be rotated to cover the functional elements and rotated to align the access openings to expose the functional element.

In another embodiment, an insertable instrument having an elongated axis associated therewith is provided that includes a first housing, a second housing rotatably connected to the first housing, at least one camera element comprising an image sensor movably connected to the first housing, at least one actuating element connected to the first housing and the camera element, and a securing element associated with the second housing for removably securing the imaging device to or against a wall of a structure having a lumen. The actuating element is capable of moving the camera element in relation to the first housing in at least one degree of freedom as described previously.

In another embodiment, the insertable instrument comprises a plurality of camera elements each comprising an image sensor movably connected to the first housing, at least one actuating element connected to the first housing and the second housing, the actuating element capable of rotating the first housing in relation to the second housing, at least one actuating element connected to each of the camera elements, the actuating element capable of moving the camera element in relation to the first housing in a first degree of rotational freedom essentially orthogonal to the elongated axis, and a securing element associated with the second housing for removably securing the imaging device to or against a wall of a structure with a lumen.

In certain embodiments, a housing of the insertable instrument has a diameter of about 5 mm (0.197″) to about 25 mm (0.984″), and is about 127 mm (5″) to about 228 mm (9″) long. In one embodiment a housing of the insertable instrument has a diameter or of 9/16″ and is about 7.8″ long. In another embodiment, the insertable instrument has a wall thickness of 0.028″. The insertable instrument may further have spherical end caps to ease insertion into the structure. An about 50 mm (2″) to about 152 mm (6″) long section of a housing of the insertable instrument may be cut away to produce an access opening that allows the functional elements to tilt when extracted. In one embodiment, the access opening is about 2.6″ long. In one embodiment, a first housing 102 has a 0.028″ thickness. In another embodiment, the first housing has a smaller diameter than the second housing which is also between about 5 mm to 25 mm and a length of about 127 mm to about 228 mm. In one embodiment, a housing of an insertable instrument has a ½″ diameter and is 6″ long, and a portion of the housing may be cut away to produce an access opening to allow the cameras to be retracted therein and extracted therefrom. A housing of the insertable instrument may include sufficient space to house cable to provide sufficient slack to accommodate the movement of the functional elements as described herein.

In another embodiment, a minimal access system is provided that includes a driving device communicatively connected to at least one insertable instrument capable of being inserted into a structure having a lumen, the insertable instrument including at least one functional element associated therewith for use during a minimal access procedure and at least one securing element for securing the insertable instrument against a wall of the structure having a lumen. In one embodiment, the insertable instrument includes at least one actuating element capable of moving the functional element in at least one degree of freedom and the driving device provides a drive signal to remotely control the movement of the functional element.

The driving device may be adapted to provide hybrid control of the insertable instrument such that the driving device may autonomously control functional element movement in at least one degree of freedom. For instance, the functional element may be a camera element and the driving device may autonomously control the camera element movement to maintain a user identified object in view while the user controls camera element movement in at least one degree of freedom to obtain an image of the site of interest from different perspectives. Additionally, the at least one functional element may be a plurality of camera elements and the driving device may autonomously control the movement of the camera elements to produce a stereoscopic image of the site of interest or to create stereo images of a site of interest in real-time based on automatic vergence algorithms.

In another embodiment, an insertable instrument is provided that includes a first housing, at least one functional element movably connected to the first housing allowing the functional element to be retracted into and extracted from the first housing, and at least one actuating element connected to the first housing and the functional element. The actuating element is generally capable of moving the functional element in relation to the first housing in a first degree of rotational freedom essentially orthogonal to an elongated axis of the insertable instrument for retracting and extracting the functional element from the first housing.

In another embodiment, the actuating element is generally capable of moving the camera element in relation to the first housing in at least one degree of freedom, such as a first degree of rotational freedom essentially orthogonal to an elongated axis of the insertable instrument, a second degree of rotational freedom essentially inline with the elongated axis, and a third degree of longitudinal freedom essentially inline with the elongated axis.

In another embodiment, a minimal access system is provided that includes a driving device communicatively connected to at least one insertable instrument that is capable of being inserted into a structure having a lumen. The insertable instrument includes a first housing, at least one functional element for use during a minimal access procedure movably connected to the first housing allowing the functional element to move in at least one degree of freedom, and at least one actuating element connected to the first housing and the functional element. The actuating element is generally capable of moving the functional element in relation to the first housing in the at least one degree of freedom. The driving device includes at least one controller that provides a driving signal to control movement of the functional element in the at least one degree of freedom.

In another embodiment, the insertable instrument includes a first housing, at least one camera element moveably connected to the first housing allowing the camera element to move in at least one degree of freedom, and at least one actuating element connected to the first housing and the camera element. The actuating element is generally capable of moving the camera element in relation to the first housing in the at least one degree of freedom. The driving device includes at least one controller that provides a driving signal to control movement of the camera element in the at least one degree of freedom, and an image tracking module that tracks movement of at least one object in a field of view of the camera element. In this instance, the controller controls movement of the camera element based on a signal from the image tracking module to maintain the object in the field of view of the camera element.

In certain embodiments, the insertable instrument may further comprise an actuating element or actuator. Various types and numbers of actuating elements or actuators for moving the functional element in relation to the housing may be used to achieve the desired degree of freedom with regard to the movement of the functional element, such as piezoelectric actuators, pneumatic actuators, solenoids, shape memory alloy actuators, linear motors, motors producing rotational movement, motors producing rotational movement adapted to provide linear movement, etc. The type of actuating element and the number of actuating elements will vary depending on the design constraints of the insertable instrument, e.g., the dimensions as dictated by the size of the access port or opening, the degrees of freedom the functional element or elements are intended to move, the number of functional element, etc. In one embodiment, at least one of the actuating elements comprises a brushless DC motor producing sufficient torque to produce the desired movement in the functional element. The DC motor may further be connected to a lead screw which when rotated can translate a shuttle or carriage in both directions along the axis of the lead screw to produce linear motion and with a bevel screw or worm gear assembly to redirect the rotational movement produced by the motor. In certain embodiments, the motor runs on 6 volts, is about 27 mm long, and has a diameter of about 5 mm.

In one embodiment the insertable instrument includes a second elongated housing that is rotatably connected to the first elongated housing, with or without bearings, such that the first and second housings may be rotated in relation to each other in at least one degree of freedom. In this instance, the insertable instrument may be removably secured to the wall of the structure having a lumen, e.g., the abdominal wall, with the securing element that is an aspect of the second housing. Tilting along the second degree of freedom may be achieved by rotating the first housing, which includes the functional element or elements therein, in relation to the second housing. The first and second housings may be rotated with respect to each other with a motor that produces rotational movement appropriately connected to each of the housings.

The insertable instrument may include, in certain embodiments, at least one electrical circuit that electrically and/or communicatively couples the functional element with a driving device. The electrical circuit may include a wire disposed within a housing of the insertable instrument. The wire can receive or communicate power, energy, sensor, video, or drive signals from a cable that interfaces with the driving device, as well as a communication unit located anywhere on the surgical device (including on the insertable instrument or trocar), which communicates wirelessly with a corresponding communication unit to provide wireless control of the insertable instrument. The wire may be adhered to a housing or integrated therein.

An insertable instrument of the present invention may also include an electrical circuit. For example, an electrical circuit may be used to: conduct a drive signal to the actuating device, conduct energy or communicate image or other data to or from the functional element, conduct energy for an ultrasonic dissector or any other type of additional insertable instrument, and receive data therefrom. In instances where functional elements may interfere with each other, such as a camera element and an ultrasound probe, the functional elements may operate at different frequencies or include some other means for limiting interference. The insertable instrument may also include a sensor, such as an oxygen sensor or oximeters, a stress/strain sensor, a temperature sensor, a pressure sensor, haptic feedback devices, etc.

The electrical circuits may be disposed longitudinally at various locations in the housing of the insertable instrument. Thus, for relatively large insertable instruments or surgical devices, all of the electrical circuits may be disposed in a localized area or areas of the first housing without limitation. For smaller insertable instruments or surgical devices, the electrical circuits may be spaced sufficiently apart around the perimeter of the housing to accommodate as many circuits as desired.

The actuating elements or the functional elements may also share electrical circuits or portions thereof. Where a plurality of camera elements communicate over the data bus, the data string or header may also associate the image data that follows with a particular camera element.

The driving device may also include an image processor/display adaptor, which receives data from at least one insertable instrument and converts image data received from the camera elements or sensors into a signal suitable for displaying the image on a monitor, such as a CRT display, an LCD display, stereoscopic goggles, etc. The image processor may receive image data from the camera elements and produces a video image of the site of interest for continuous video display. With other types of data acquisition elements, the system may convert the signal received from these elements into a numerical or graphical representation of the signal for display. For example, the system may convert a signal from a pressure sensor into a numerical value. The image processor may also process the image data for other purposes, such as to extract data from the image data. The extracted data may represent an object or a portion of the object in the field of view, which may be used to track the object as discussed below.

In one embodiment, the system provides hybrid control, which allows the user to control movement with regard to some of the degrees of freedom of the insertable instrument while the system autonomously controls movement with regard to the others. For example, the system may be adapted to autonomously control camera movement in the first and second degrees of freedom in order to keep a user-identified object in view, while the user controls camera movement along the third degree of freedom to provide images from different orientations/perspectives. In certain embodiments, the autonomous system maintains the user identified object (such as an organ) in view while the user orients at least one camera element. This may be accomplished with a constraint-based sensor planning system that can associate viewpoints of modeled objects. The planning system generally incorporates constraints on viewpoint visibility, depth-of-field, and image resolution to plan correct viewing parameters and positions. This aspect is particularly beneficial when multiple camera insertable instruments are in use to provide surgeons with a choice of potential viewpoints and to provide stereoscopic imaging.

The system may also independently track user-identified objects to maintain such objects in view when the objects move in the site of interest or more particularly in the image field. For example, the system may track the movement of organs or instruments in a subject's abdominal cavity and control the camera element to maintain the organ or instrument in view during a procedure. This may be accomplished with a tracking module, which receives a first set of image data of the site of interest and instruction regarding an object or objects to be tracked, which object or objects are represented in the first set of image data. A set of image data generally includes data sufficient to identify an object in the field of view. The set of image data may include sufficient data to produce an image or frame of a video or a subset of such data. The user defined targeting instructions may be received with a pointing device (such as a mouse, a joystick, a stylus, a touch screen display) that allows the user to select an object or point on a graphic display of the site of interest. The tracking module receives a subsequent set of image data and tracks movement therein of the user-identified object based on differences between the first set of image data and the subsequent set of data. Accordingly, movement may be tracked in real-time based on a comparison of contiguous and/or sequential image data sets or frames obtained at different times. The image data sets may be stored in a data store associated with the tracking module for tracking or for reproduction at a later time.

The tracking module may operate on many different imaging cues, such as gray-level regions, geometric features, motion, fiducial markers, etc. In one embodiment, image processing is used to identify a target based on its RGB color components.

Such insertable instruments as those described above are further described in the parent patent and application to the present application, now issued as U.S. Pat. No. 7,066,879 to Fowler et al. and published as U.S. Patent Publication No. 2007/0032701 to Fowler et al., respectively, hereby incorporated by reference in their entireties.

The insertable instrument may be, in various embodiments, an instrument that is purely manual, or an instrument that requires a power supply. In certain embodiments, the insertable instrument may be powered via an external power supply connected by a cord attached to an electrical outlet, or may be powered portably, as, e.g., by battery. For example, certain insertable instruments contemplated by the present invention may be powered by alkaline, lithium, nickel-cadmium or other batteries known in the art, and may be rechargeable. Additionally, the insertable instrument may be powered by magnetic induction, nuclear, fluid dynamic, solar, or similar energy sources; as well as by external control, e.g., remote control (using, for example, an infrared receiver and LED light pulses).

In various embodiments, a insertable instrument of the present invention further comprises a sensing chip such as, for example, a charged coupled device (CCD) (in one embodiment, the CCD image sensor is mounted in a lens housing having threads therein to accept the lens and to accommodate focal adjustments), charge injection device (CID), photo diode array (PDA), complementary metal oxide semiconductor (CMOS) chip or a light sensing chip; or, for example, a CCD color image sensor mounted essentially perpendicular to a 17 mm long driver board, and the driver board is electrically connected to a camera control unit (“CCU”) remote from the insertable instrument. These portions of the insertable instrument may be inserted into the body of the patient when the surgical device is used in a procedure. In these and other embodiments of the present invention, one resultant advantage is less “crowding” of instruments through each incision.

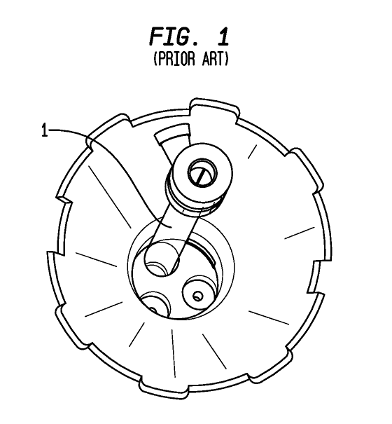

Turning now to the Figures, FIG. 1 illustrates a device known in the art, specifically, the pNavel® device for single incision surgery. This device includes a port for a laparoscope 1.

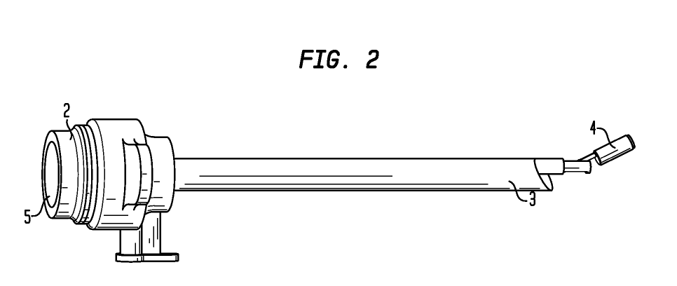

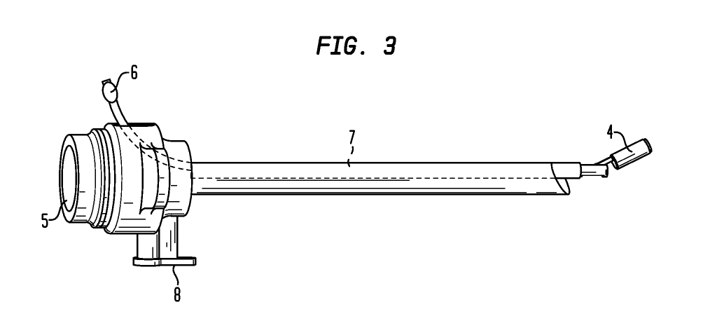

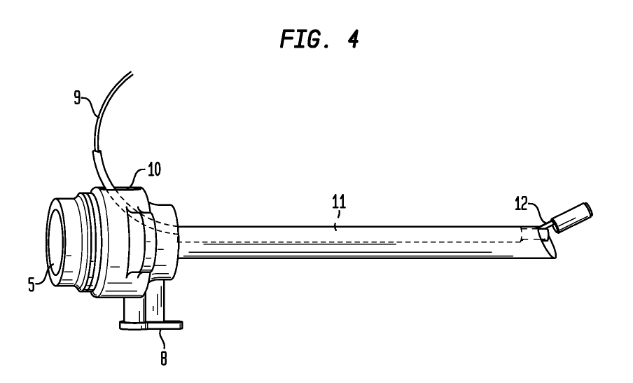

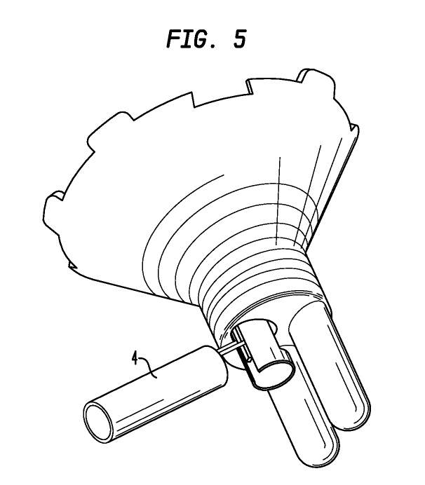

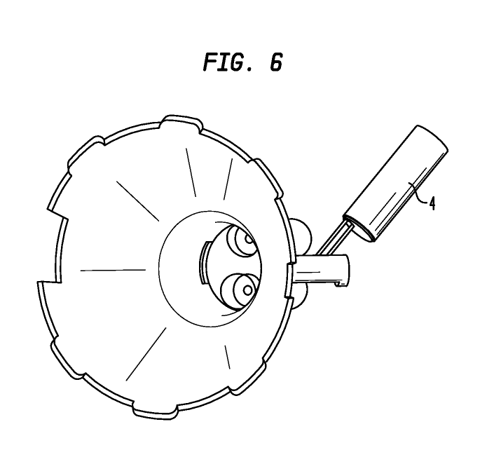

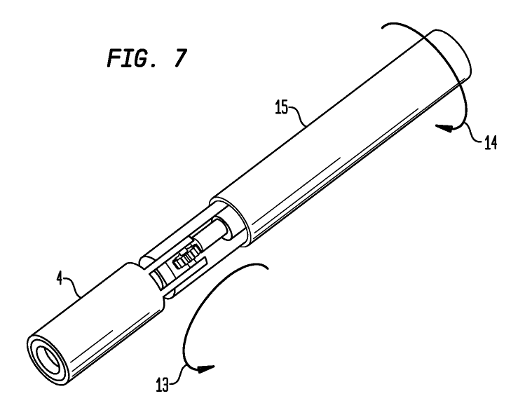

As shown in FIG. 2, a insertable instrument/trocar combination of the present invention comprises an external end 2 comprising one or more orifices 5 for receiving instruments and an insertion end 3 comprising a camera module 4. FIG. 3 illustrates another embodiment of the present invention, wherein the insertable instrument/trocar combination further comprises a knob 6 for mechanical control of the camera pan and tilt, one or more cables/pulleys 7 for control, and a CO₂ port 8. As shown in FIG. 4, the insertable instrument/trocar combination may further comprise one or more of the following: a motor 10, a cable of wires 9 that connects the insertable instrument/trocar combination to a computer or power source, or additional devices; one or more gears or screwdrivers 11 for control of the camera; or a worm gear 12. As used herein, the term “worm gear” means a combination of gears that will allow changing the angle at which the force is delivered—in various embodiments, an angled joint through which the rotation of a screw drive's direction can be changed. FIG. 5 further shows another embodiment of the present invention. FIG. 6 shows a side view of another embodiment of the present invention, where the interior of the trocar can be seen, and wherein the camera module 4 is displayed as it may be during use. FIG. 7 shows a insertable instrument according to certain embodiments of the invention, comprising a camera module 4 and a pan/tilt platform 15 that can be rotated in various directions to control the pan 13 and tilt 14 of the instrument.

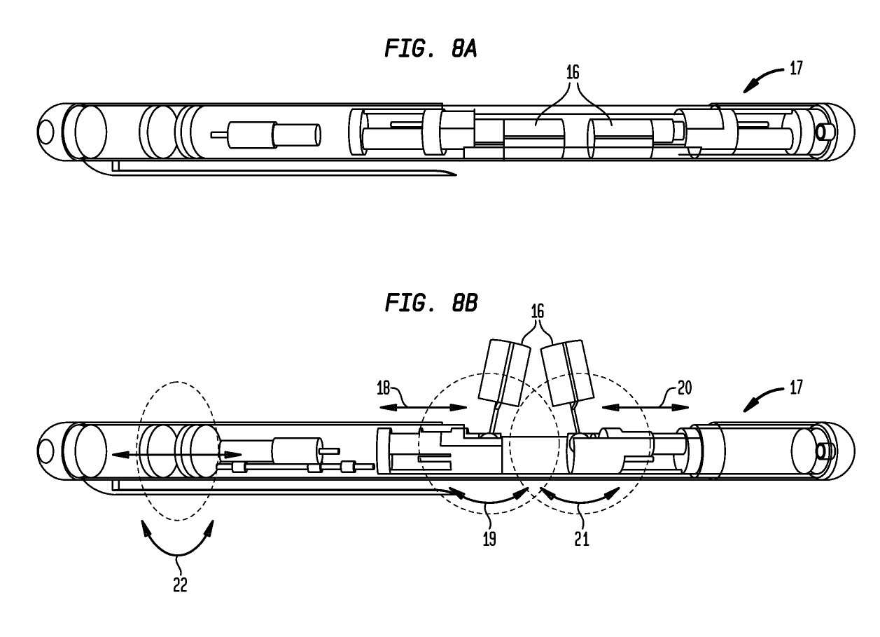

FIG. 8(a) shows another embodiment of the present invention, wherein the insertable instrument 17 comprises imaging devices 16, shown retracted. FIG. 8(b) shows the embodiment wherein the imaging devices 16 are extracted and the insertable instrument is engaged, as it would be after insertion into the body of a patient. The two imaging devices 16 on the insertable instrument 17 can be rotated in a left translate 18 and left pan 19 direction, and a right translate 20 and right pan 21 direction. The entire imaging device can also be rotated in a combined tilt 22 direction.

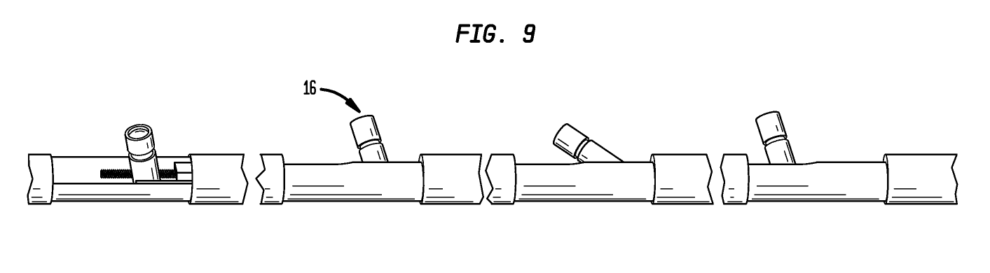

FIG. 9 shows another embodiment of the present invention, wherein the insertable instrument is an in vivo imaging device having a single camera, but wherein the platform can be designed to contain two cameras.

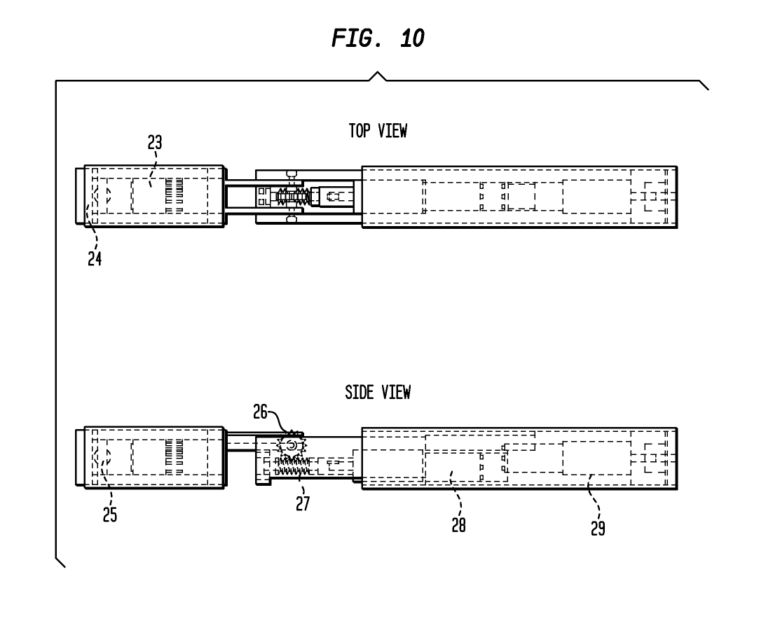

FIG. 10 shows another embodiment of the present invention, wherein the insertable instrument is an in vivo imaging device, as described in Example III. In FIG. 10 this imaging device is shown from both a top view and a side view. The imaging device contains a CCD sensing chip 23, and LED light source 24, a camera with a lens 25, worm gear 26, a worm 27, a pan motor 28 and a tilt motor 29.

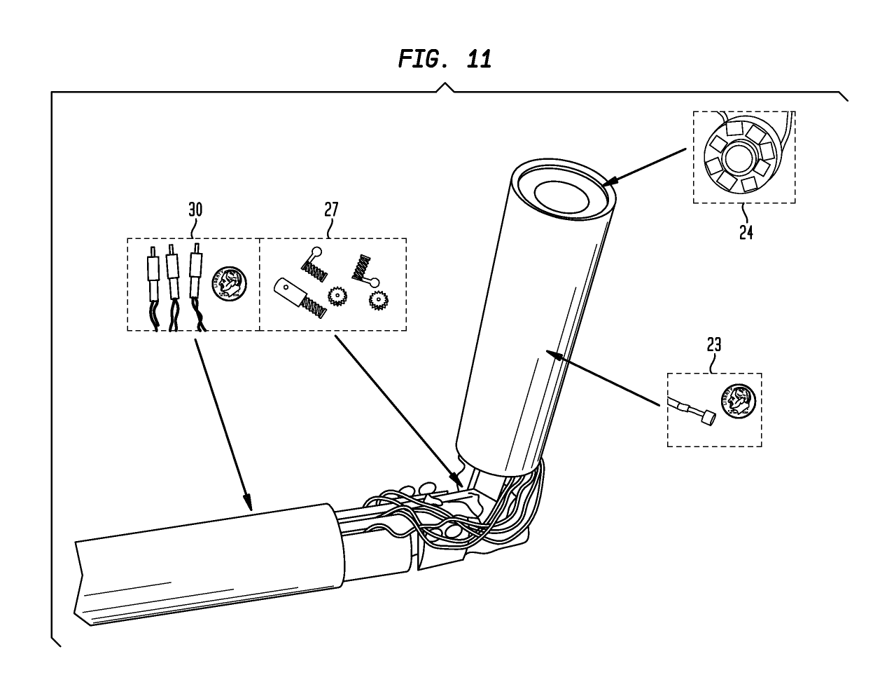

FIG. 11 shows a insertable instrument in accordance with another embodiment of the present invention device axis is shown, with a close-up of the CCD sensing chip 23, and LED light source 24, the motor 30 and the worm 27. This is typical of a insertable instrument according to the present invention and further described in Example III.

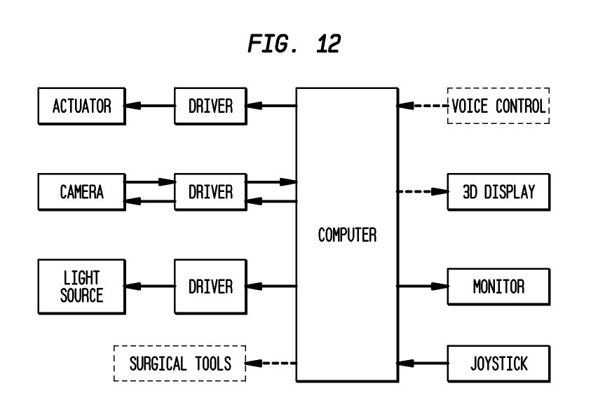

FIG. 12 is a depiction of the overall system configuration of the embodiments described in Example III.

### Imaging Device

In certain embodiments, the insertable instrument comprises an imaging device. As used herein, “imaging device” means the imaging elements (including hardware and software) and circuitry used to produce a video signal that can be accepted by a standard video device such as a television or video monitor accompanying a computer. The imaging device may be of any type known and used in the art of patient procedures; for example, it may comprise an opto-electronic viewing system such as that shown in U.S. Pat. No. 5,944,713 to Schuman, hereby incorporated by reference in its entirety. In various embodiments, the imaging device comprises a microscopic or spectroscopic imaging device, or comprises a camera.

In certain embodiments, the trocar may be designed to allow independent movement of a camera in relation to the instruments within the trocar. The portals on the surgical device may be configured to swivel within the trocar to which the imaging device is attached. Alternatively, gel technology may be used, such that the instruments may move externally within a soft gel matrix while the imaging device is attached to the trocar, which may be, for example, a fixed ring placed internally within the body of the patient.

The imaging devices of the present invention may, in various embodiments, be movable on one or more of the following: a pan axis, a tilt axis, a translation axis and a zoom axis. The imaging device may be, for example, camera having up to three degrees of freedom: pan, tilt and translation. In certain embodiments, the imaging device disposed on the insertable instrument may have up to five degrees of freedom. The control of at least one of the pan axis, tilt axis translation axis and zoom axis may be operated either manually (as in, by the surgeon's hand or foot pedal, or by an operator), or in conjunction with mechanical control such as cables or wires; or remotely (as in via remote control, with an infrared receiver and an LED transmitter).

In certain embodiments, the imaging device comprises a maneuverable camera, and the camera may be located anywhere on the imaging device that enables it to image or scan the interior of the patient's body, depending upon the specific purpose of the procedure. By using the pan/tilt axes as described herein, the imaging device can provide a larger viewing volume than traditional devices, which are restricted by the fulcrum point of insertion. The camera may be located on a distal end of the imaging device, or anywhere else along the surface or interior of the imaging device or trocar.

In various embodiments of the present invention, the device may further comprise one or more apparatuses such as a tip that emits laser or infrared radiation, light emitting diodes (LEDs), other tips that can emit radiation of a specific frequency (spectroscopy), or ultrasound for diagnosis, imaging, guidance or treatment. Light emitting diodes (LEDs) have been used as light sources for medical devices in the past, and have the advantage of low power and cost, high efficiency, small package size and long lifespan. The Luxeon Portable PWT white LED (LXCL PWT1) may be useful as the illumination unit in certain embodiments of the present invention. It has a small package size of 2.0×1.6×0.7 mm, which can generate 26 lumens of light at 350 mA, has a color temperature of 6500K, and a lifespan of about 2,000 hours. In certain embodiments, the surgical device may comprise an illumination unit including a custom made PCB (printed circuit board) board having 8 LEDs. Its size is 9 mm in external diameter, 5 mm in internal diameter, and 3 mm in thickness. The 8 LEDs are serially connected and soldered in a circular printed circuit board. It can deliver a total of 208 lumens of light, with a power consumption of merely 8.4 W.

In various embodiments, the imaging device of the present invention may comprise more than one camera; for example, certain embodiments of the present invention are directed to a insertable instrument/trocar combination comprising two cameras disposed along a scope. In some embodiments, a surgical device according to the present invention comprises two cameras, both having a common tilt axis, and each having independent pan and translation axes. In certain embodiments, the first camera is capable of tilting about the central device axis, panning about an orthogonal axis, and translation, and the second camera has a common tilt axis as the first camera, but independent pan and translation axes.

In certain embodiments, the imaging device may or may not be totally blind; that is, the imaging device may be working during insertion, and can be used to find attachment points within the interior of the patient's body.

An imaging device according to the present invention may comprise a cable of wires for power and image transmission, or may be wireless. The imaging device may additionally be monoscopic or stereoscopic. The size of the imaging device itself may be as small as a fraction of a millimeter in diameter (e.g., about 0.2 mm, about 0.3 mm, about 0.4 mm or about 0.5 mm) or as large as about 2 centimeters or more. In various embodiments, the imaging device should be small enough such that when it is attached to the trocar, the resultant combination enables perform minimally invasive surgery using a single incision in the skin of the patient. By using a very small chip, the imaging devices of the present invention are useful for any minimal access surgery applications for a patient, e.g., arthroscopic procedures.

In certain embodiments, an imaging device of the present invention may comprise an illumination device such as a light, for example, an LED, an infrared light source, or external light source delivered by fiberoptics or the like. Light of any valuable frequency (infrared, ultraviolet, spectroscopic examination using specifically generated or filtered frequencies of light) may be useful for the devices and methods of the present invention.

Video signals from the imaging device may be transmitted in any format (for example, NTSC, digital, PAL and the like) that can be received and broadcast on a video monitor for viewing by a surgical team. The devices and methods of the present invention may utilize solid state imaging technology, such as CMOS imaging, MOSAD (multiplexed over sample analog to digital) technology, computer monitors based on cathode ray tubes, LCD or plasma displays and the like.

### Trocar

The trocar to which the insertable instrument is attached may be any size or type of trocar that is capable of being inserted into an opening in a patient's body to maintain an opening of a desired diameter for ease of a procedure. In certain embodiments, the trocar is capable of retaining a tension pneumoperitoneum when inserted into portions of the patient's body, e.g., the abdomen, for procedures such as surgery. In certain embodiments, the trocar may use gels, elastic polymers, simple or complex reducers appropriate for the size of the instrument(s) that pass through the trocar, or any other mechanism to retain the pneumoperitoneum without leak. In other embodiments, the trocar need not retain pressure or retain a tension pneumoperitoneum. For example, trocars for placement in the chest of a patient generally do not need to retain a pressurized internal environment when placed into the opening in the patient's body.

In various embodiments, the trocar may have one or more entry sites for up to several insertable instruments, such as that described in U.S. Patent Publication No. 2008/0255519 to Piskun et al., directed to a laparoscopic port assembly comprising a port having multiple tubular port members projecting therefrom.

In certain embodiments, the insertable instrument/trocar combinations of the present invention may comprise any form of trocar know in the art. In particular, a preferred trocar for the embodiments of the present invention may be one of standard shape and size, such as a simple tube slipped into the incision in the skin and used to hold it open, or a specialized trocar adapted for specific uses; for example, a trocar having extensions on its lower portion that are inserted into the incision and then expanded internally to hold the incision open by contact with the inside surface of the patient's body, whether or not adjacent to or near the point of the incision, while the upper portion of the trocar rests on the outside surface of the patient's body.

In yet other embodiments, the trocar may be an elongated tube that resembles a syringe, in that it is injected into the skin of the patient through the incision, and the insertable instrument, in addition to being attached to the trocar, is further threaded into the patient's body through the trocar's elongated body. The connection between the insertable instrument and the trocar may be permanent or temporary through the life of the procedure that is, the insertable instrument may be attached to the trocar either before, during or after insertion of the surgical device into the patient.

In certain embodiments, the trocar is capable of maintaining an opening in the surface of the patient that has a diameter of about 3 cm or less, about 2.5 cm or less, about 2 cm or less, about 1 cm or less, about 1 cm to about 3 cm, about 1.5 cm to about 2.5 cm or about 2 cm to about 3 cm. In various embodiments, the trocar is capable of maintaining an opening in the surface of the patient that has a diameter of about 0.1 mm to about 30 mm, about 0.2 mm to about 20 mm, about 0.3 mm to about 15 mm, about 0.5 mm to about 12 mm, about 1 mm to about 10 mm, about 5 mm to about 10 mm, about 10 mm, about 12 mm or about 15 mm. In any event, the diameter of the opening created by the trocar should be sufficient to insert any of the insertable instruments described in the present disclosure.

In certain embodiments, one or both of either the trocar or the insertable instrument may be reusable or single use, recyclable or disposable, such that any of the foregoing may be interchangeable with any of the other foregoing.

### Attachment of the Insertable Instrument to the Trocar

As used herein, the term “attached” means joined to or connected to, as used to describe mechanical devices. In various embodiments, the surgical device may be either reversibly attached or irreversibly attached to the trocar. In various embodiments, “attached” may be used to refer to a situation in which surgical device and trocar are bound together so closely that they are essentially integrated into a unified device.

In certain embodiments, the insertable instrument is attached to the trocar via any one of the following attachment means: adhesion, a hook, a magnet, a clamp, a snap, welding, a thread or string, a wire, soldering, a pin, a screw, threading or a plastic connector.

For example, the insertable instrument and trocar may be attached via adhesion. Such adhesion may be achieved by, for example, application of an adhesive (for example, an adhesive strip or patch, or direct application of an adhesive material such as a glue, paste, liquid or gel to one or more of the surfaces of either the trocar or the insertable instrument). Useful adhesives include any that are typically used in the art of medical devices to connect instruments to each other or to hold them in place. In addition, some examples of adhesives that have been found to be useful in adhering body tissues during or after surgical procedures include fibrin sealants, cyanoacrylate sealants, gelatin and thrombin mixtures, polyethylene glycol polymers, albumin and glutaraldehyde tissue adhesives (for example, adhesives based on bovine serum albumin). In addition to their ability to adhere tissues to each other, such adhesives may additionally be advantageous for attaching an insertable instrument to a trocar, in accordance with certain embodiments of the present invention.

In certain embodiments, the insertable instrument and trocar may be attached to each other by bonding of metal, for example, welding, soldering, riveting or brazing specifically, in certain embodiments, where both the insertable instrument and the trocar comprise metal, both can be bonded by applying heat to melt the metal, applying pressure to the point of attachment, or a combination of both heat and pressure. In other embodiments (such as soldering and riveting), the two pieces of metal are joined via a third piece of metal. Any of these methods of bonding metal are contemplated within the scope of the invention.

In certain embodiments, the insertable instrument and trocar may be attached to each other via a hook and loop system, wherein one of the insertable instrument or trocar has a hook along its surface, and the other has a corresponding loop that attaches to the hook, thereby attaching the two but permitting a range of motion without detachment (a simple hook is depicted on the end of an instrument shown in U.S. Pat. No. 5,397,333 to Knoepfler).

In other embodiments, the insertable instrument and trocar may be attached via mechanical loop fastening, which is similar to a hook and loop system, but may involve two permanently or semi-permanently linked and closed hooks, similar to the links of a chain; or a ferruled loop surgical fastener, as described in U.S. Pat. No. 5,879,371 to Gardiner et al.

In other embodiments, the insertable instrument and trocar may be attached by snap or button—specifically, two mating parts are separated before or after the surgical procedure, and one part attached to the insertable instrument and trocar respectively. Bringing the two mating parts together (either via snap or buttonhole) will attach the insertable instrument and trocar. This may be done before the procedure begins, during the procedure to anchor the insertable instrument temporarily, or after the procedure for separation of the two and ease of removal.

In other embodiments, the insertable instrument and trocar may be attached by electrostatic force, that is, the charge exchange between the molecules on the surfaces of each of the insertable instrument and the trocar, wherein at least one has a surface exhibiting a high resistance to electrical flow. In such a case, the charge may be neutralized at any point in the surgical procedure to separate the insertable instrument and the trocar.

In other embodiments, the insertable instrument and trocar may be attached by tying a wire or string or thread or plastic connector; that is, where at two may be simply bound together with such material, while still allowing for freedom of motion. In such embodiments, the attachment is likely to be completed prior to the beginning of the surgical procedure, as tying the insertable instrument to the trocar may be difficult to achieve during the procedure, while the surgery is being performed. In alternate embodiments, the connection may be made by plastic or polymeric connector or anchor similar to those used in a variety of instruments as plastic “ties” or “handcuffs” for lashing together mechanical parts or packaging.

In other embodiments, the insertable instrument and trocar may be attached by pin with a socket or catch (for example, a needle that is threaded or notched, or a brad). This is similar to attachment with a loop and hook, except that the attachment may be more permanently or strongly fixed, and may in certain embodiments allow for greater range of motion of the insertable instrument. The pin may, in certain embodiments, be further attached to a chain, rope or other elongated material that allows the insertable instrument to move freely within the interior of the patient's body.

In other embodiments, the insertable instrument and trocar may be attached by screw, nail or staple. For example, a screw, nail or staple may be used to connect the insertable instrument and trocar at a point that is fixed or movable. In some embodiments, the insertable instrument may comprise several joints, wherein one joint is fixed to the trocar via a screw or nail, and the remaining joints move freely. In other embodiments, the insertable instrument may comprise a single joint that is loosely screwed or nailed to the trocar, allowing for a wide range of motion. In other embodiments, as described above, the trocar may have a portion comprising threading, such that insertable instrument may be attached to the trocar by threading into the patient's body through or alongside the trocar's elongated body.

In other embodiments, the insertable instrument and trocar may be attached by a magnet, i.e., application of a magnet on the surface of either the trocar or the insertable instrument, or both, to keep the two attached to each other before, during or after the procedure; or, in the case where one of the insertable instrument or trocar is metallic but the other is not, then application of a magnet to only the surface of the non-metallic of the two. Alternatively, one or both of the insertable instrument and trocar may be comprised of magnetic material. The magnetic field may be created by the nature of the material itself, or may be adjustable (e.g., electrically-induced). The advantage of using a magnetic connection is that it can easily be detached by the surgeon as needed during or after the procedure, either by pulling the parts apart manually or by switching the magnetic field on and off

### Attachment of the Devices to the Interior of the Patient's Body

In various embodiments, a detachable introducer, such as, e.g., a rod, may be used to introduce the insertable instrument to the body of the patient. Once in position, the insertable instrument or the insertable instrument/trocar combination may further comprise one or more clasps, which may be released from the body of the instrument or the insertable instrument/trocar combination to pierce the wall of the body surface enough to hold the camera thereto; for example, to the abdominal wall of the patient in the case of an abdominal procedure, or to the walls of any other surface of the patient for a corresponding procedure (e.g., the walls of another organ, blood vessel or the like). Once the clasps are in place on the wall of the body surface, the detachable introducer may be detached and removed from the body of the patient. The rod may comprise cabling for this purpose. In various embodiments, the rod may be of about 2 to about 5 mm diameter, about 2.5 to about 4 mm diameter or about 3 mm diameter; and may be made of any of a variety of materials including metal, wood, plastic or other polymeric material, rubber or ceramic. At the end of the procedure, the clasps may be reattached for removal of the insertable instrument or the insertable instrument/trocar combination from the body of the patient.

In various embodiments, the insertable instrument/trocar combinations of the present invention may be configured when in use such that one or more portions of the insertable instrument are fixed to the wall of the patient's interior, for example, fixed to the wall of the patient's abdomen. In various methods according to the present invention, an external holding mechanism is used. This mechanism may have a rotational attachment that holds the tilt motor end of the insertable instrument/trocar combination.

In various embodiments, in order to facilitate a procedure through a single incision, other ways are available to fix the devices of the present invention to the patient's body at points such as the walls of the abdomen. For example, an external holding mechanism may be used. Such a mechanism may have a rotational attachment that holds a tilt motor end of the device. When the surgeon grasps the handle of the mechanism, this attachment may rotate up to about 90 degrees when the device is deployed into the patient's body. The surgeon can then pull the handle and rotate the device to the point where it is up against the desired inner surface of the patient's body, e.g., the abdominal wall.

One example of such a mechanism is discussed in U.S. Pat. No. 7,429,259 to Cadeddu et al., which discusses a surgical anchor system for holding a surgical tool in place. The present devices and methods are advantageous over those taught by Cadeddu et al. because they are capable of providing, in various embodiments, stable surgical devices that comprise an insertable instrument attached to a trocar, without the need for any additional supports such as an external anchor. Thus, the presently claimed devices and methods are easier to assemble, use and disassemble, in addition to being more economic. Moreover, they provide the additional advantage of being, in certain embodiments, recyclable or sterilizable for repeated use.

In certain embodiments, a spring loaded needle and suture system may be affixed to a device of the present invention, and can be activated, either mechanically or electrically, to puncture the lumen in the patient's body from the inside to bring the suture to the outside of the patient's body, where it can be used to fix the device to the lumen. For example, the spring loaded needle and suture system may be used to puncture the abdomen from the inside to bring the suture to the outside of the patient's body, where it can be used to fix the device to the abdomen.

A further possibility is to use magnetic anchoring, as discussed by Park et al., “Trocar-less Instrumentation for Laparoscopy,” Surgical Technique 379-384 (March 2007), the contents of which are hereby incorporated by reference in their entirety. As examples of such a configuration, two internal magnetic attachments, such as magnetic pads, can be installed on the insertable instrument. When it is fully deployed into the body of the patient, the surgeon can use external magnetic components to maneuver the locomotion of the insertable instrument within the body from a corresponding spot outside of body. An advantage of this method is it is non-invasive; however, the intensity of the magnetic field may vary with variations of the thickness of the patient's skin and tissues.

In certain embodiments, the invention is directed to a method of forming an insertable instrument/trocar combination comprising: inserting a trocar into the interior of a patient's body, inserting an insertable instrument into the interior of a patient's body, and attaching the insertable instrument to the trocar via a point of attachment contained within the interior of the patient's body. In use of the resultant insertable instrument/trocar combination, each of the insertable instrument and the trocar may be, in various embodiments, either disposed entirely within the interior of the patient's body, or partly outside the patient's body.

In certain embodiments, the invention is directed to a method of performing a surgical procedure comprising inserting an insertable instrument/trocar combination. The method may further comprise removing the insertable instrument/trocar combination after the surgical procedure.

When the insertable instrument/trocar combination is fully in use, in various embodiments the camera may comprise an actuation mechanism for engaging or moving the insertable instrument. In various embodiments, this actuation mechanism may or may not be wholly inserted into the body of the patient when the insertable instrument is inside the body of the patient; that is, the actuation mechanism may be wholly inserted, or it may be within the insertable instrument, such as, e.g., within the trocar in the part of the trocar that remains outside of the body of the patient, or it may be detached and separate from the trocar. The actuation mechanism may be mechanical or electrical (e.g., operated by motor, remotely controlled, computer controlled with algorithms and the like).

As is the case with any instrument that is associated with surgery or other procedures and/or inserted into the body of a patient, any or all parts of the surgical devices of the present invention are capable of being sterilized. In various embodiments, any of these insertable instruments and trocars are sterilizable by any known sterilization technique, e.g., autoclave, contact with a disinfectant (e.g., soaking, dipping, coating, spraying), ultrasonic sterilization, application of a vacuum, dry heat, gas plasma sterilization, cold sterilization, infrared radiation, and the like.

The surgical device/trocar combinations may be engaged by motors that are controlled remotely such as mechanically by the surgeon or another operator, or by other means, e.g., by the use of computer software. In various embodiments, control of the movements of the devices of the present invention in any part (including the entire mechanism or just the image source) may be via a computer, with any other human interface including but not limited to a joystick, a keyboard, voice control using voice recognition software, or mechanical control, e.g., push-pull mechanisms, clamps, graspers, forceps, foot pedals, pulleys or gears and the like.

The devices and methods of the invention provide advantages over instruments known in the art because, among other reasons, their use in a procedure can eliminate the need for a separate insertable instrument, and may facilitate single-incision surgery by allowing more space for other instruments through the port, and/or may facilitate multiple-incision surgery by eliminating the need for at least one trocar, thus minimizing the number of incisions and decreasing the chances of infection and shortening recovery time for the patient.

In various embodiments, the incision has a length of about 2 to about 12 mm, about 5 to about 10 mm or 10 to about 30 mm, depending on the type of surgery, the size of the cavity and the size of any specimen requiring removal from the patient's body.

The methods of the present invention may be directed to any procedure that can be performed on a patient; for example, surgery, an exploratory or diagnostic procedure, treatment of an interior cavity; repair of an interior surface of a patient (e.g., organ surfaces or walls), removal of an internal organ, tissue, foreign object or material such as bone, blood, or infectious matter from a patient's body; scanning an interior surface of a patient; or delivery of an agent, e.g., radiation, contrast or imaging agents or medication to the interior of a patient.

The interior of the patient's body may include any surfaces comprising the patient's cells; for example, the internal organs, the surfaces thereof (including for example, the linings of hollow organs), the contents thereof (including digested or undigested food or foreign bodies), tissues, blood vessels, body cavities, tumors, or any other materials that may be present in the body of a patient.

The insertable instrument/trocar combinations of the present invention may comprise a trocar attached to any of a number of insertable instruments known in the art. Any of a number of insertable instruments can be attached to a trocar to provide surgical devices and methods in accordance with the present invention. The devices of the present invention may comprise additional components such as, for example, additional insertable instruments, such as sensor devices, e.g., sensors for temperature, pH, the presence of various gases, electrical potential, other conditions in the patient's body such as heart rate, blood pressure, respiration, specific radiation frequency (spectroscopy), humidity, moisture content and the like.

In certain embodiments, the surgical device of the present invention may comprise a robot for surgical applications comprising manipulator arm means, the arm means comprising first and second arm components pivotally connected to each other and capable of being fully inserted within the body of a patient, as well as methods of performing surgery using such a device, such as those described in U.S. Pat. No. 7,492,116 to Oleynikov et al.

In certain embodiments, the present invention is directed to surgical devices that are comprised of part or all of a robot such as a mobile mini robot having a small size, translational mobility component with a motor and a controller component coupled thereto, configured to apply translational pressure on any surface; as well as such robots comprising a single axle. Such robots may be able to move forward, backward, and even turn in circles. Such devices may further contain sensors that sense pH, temperature, gases, fluid composition, respiration rate or humidity, as described in U.S. Pat. No. 7,199,545 to Oleynikov et al.

Another example of a surgical device in accordance with the present invention is a device comprising a miniature camera robot that can be placed entirely within an open space such as an abdominal cavity, having adjustable-focus camera means, pan or title camera means, and support means (such as a support component for supporting the robot body), and a light source for illumination and a handle to position the robot. Such a miniature robot may further be produced from material selected for being sterilizable, and may further contain sensors to measure parameters such as temperature, pressure, the presence of various gases and/or humidity, and may be adapted to fit through the port of a laparoscopic tool to obtain an internal image of an animal. Such a miniature camera robot is described in U.S. Pat. No. 7,339,341 to Oleynikov et al. and U.S. Patent Publication No. 2008/0058989 to Oleynikov et al.

Another example of a surgical device in accordance with the present invention is a device comprising a mobile robot having an agent delivery component and an agent reservoir, a translational mobility component and an optional rotation component. Such a device may further comprise: a power source, an actuator, a controller component coupled thereto, a mixing and discharge component in fluidic communication with the agent reservoir, such as a delivery tube; a manifold in fluidic communication with the at least one delivery tube; and a cannula in fluidic communication with the manifold. Such a mobile robotic device is further described in U.S. Patent Publication No. 2009/0069821 to Farritor et al.

Another example of a surgical device in accordance with the present invention is a device comprising a mobile microrobot for surgical applications such as collecting biopsy samples, having a mobilization element comprising two wheels and various sensors to measure temperature, blood or other fluids in tissue, electrical potential, heart rate, fluid composition, respiration rate, humidity, pressure or pH; as well as any of the following: tranceivers, imaging capability, or one or more manipulators. Such a microrobot may further comprise a member disposed between the two wheels and extending from the body in a direction substantially perpendicular to the axis of rotation of the two wheels for converting rotational motion of the wheels into translational motion; or a manipulator arm. Such mobile microrobots are further described in U.S. Pat. Nos. 7,042,184 to Oleynikov et al., 7,372,229 to Farritor et al. and 7,126,303 to Farritor et al.

The materials and methods described herein exhibit many advantages over those currently available. All of these advantages, as well as others that may become apparent upon use, will lead to significant reduction of costs and waste.

Various embodiments of the invention will be more fully described herein in the Examples.

### Example 1

### Prototype I

The prototype created is a fully insertable, micro robotic imaging platform for minimally invasive surgery as a replacement for conventional laparoscopes and endoscopes. The product offers multiple degrees-of-freedom (pan, tilt and zoom movement) with picture-in-picture imaging, automatic instrument tracking and alternative joystick control.

| | |
|---|---|
| (a) Dimensions: | Length of 110 mm and width of 11 mm insertable through standard |
| | 12 mm trocar |
| (b) Housing (Shell) Material: | Stainless steel |
| (c) Components: | + Camera head - ¼″color video with active pixels 752(H) × |
| | 582 (V) at PAL System - 450 TV lines (H) × 420 (V) |
| | + Camera head 6.5 mm in diameter |
| | + Lens - miniature pin-hole lens machined to 6.5 mm o/s dia. |
| | (focal length 5.0 mm and F number 4.0) |
| | + Pan/tilt mechanism |
| | − Brushless DC motor |
| | − 625:1 Planetary gear head |
| | − Dimensions: 27 mm × 5.8 mm diameter |
| | + Focusing range: 40-100 mm. |
| | + Zoom: Digital via software |
| | + Worm gear --- 16:1 reduction ratio (machined to 125:1) |
| | + Motor driver board |
| | + Integrated LED light source (custom printed circuit board) --- |
| | 8 LEDs (9 mm ext. dia/5 mm int. diameter/3 mm thickness) |
| | + Control interface driver |
| | + PC (Dell) |
| | + Joystick controller |
| | + 2-D and 3-D format (latter 2 camera heads) |
| | + Additional software: Automatic color feature tracking, |
| | visual servoing to track features, image processing |
| | (rotation, picture-in-picture, zoom, lens distortion |
| | correction) |
| (d) Additional Details: | |

List of Components:

| | | |
|---|---|---|
| Component | Supplier | Product |
| Lens | Universe Kogaku America | Minature pin-hole |
| | | PTS 5.0 |
| | | [Machined to 6.5 mm o/s diam.] |
| Camera | NET USA, Inc. | ¼″ Color Video |
| | | (Part # CSH 1.4-V4-END-R1 |
| | | Active Pixels 752 (H) × 582(V) |
| | | @ PAL System |
| | | [Provides 450 TV lines (H) |
| | | and 420 TV lines (V)] |
| Sapphire | Edmund Optics | 9.5 mm |
| Pan/Tilt Mechanism | Smoovy, Inc. | Brushless DC motor |
| | | (Part #0513G) |
| | | 625:1 Planetary gear head |
| | | Series OGA |
| | | Length: 27.0 mm × 5.8 mm diam. |
| Worm Gear | Kleiss Gear, Inc. | 16:1 gear reduction ratio |
| | | [machined to 125:1 gear ratio] |
| Motion Control Board | National Instruments | NIDAQ PCI-6713 Board |
| | | with SCB 68 |
| | | Breakout Board |
| Motor Drivers | Smoovy, Inc. | BLCPS.00028 |

### Example II

### Prototype II

Another insertable instrument, as depicted in FIG. 8, available for attachment to a trocar within the scope of the present invention, is an imaging device whose outer shell is a tube 22 mm in diameter, 19 cm long, and has cabling emerging from its proximal end. The device contains a first motor that controls the tilting motion of the cameras, and is parallel with the central axis of the shell and near the proximal end of the device. The motor rotates in an inner shell that contains both cameras and additional motors. A 5.8 cm long section of the outer tube is cut away at the distal end to allow the cameras to tilt 180° when they are extracted. Visual serving experiments with Prototype II demonstrate that the system is capable of keeping a moving target pattern within its field of view. Image-based visual serving was used to track the target automatically.

In suturing experiments with a laparoscopic training box, the insertable instrument can provide an image that is sufficiently clear to perform the task without using any additional image sources. The user or an assistant can adjust the view by joystick control of the camera's pan, tilt and translation axes. The insertable instrument exhibits desirable results, such as high quality imaging and ease of use, when tested in a mockup using a surgical training box in accordance with Strong et al. 2005 (with a training box designed for MISTELS: McGill Inanimate System for the Training and Evaluation of Laparoscopic Skill, as described in the publication Derossis A M, Bothwell J, Sigman H H, Fried G M: The effect of practice on performance in a laparoscopic simulator, Surg Endosc 12:1117-1120, 1998.

### Example III

### Prototype III

Another insertable instrument is available for attachment to a trocar within the scope of the present invention. The insertable instrument is smaller than that of Example II, and it includes an integrated light source. The surgical device has a two motor design with pan and tilt axes for a single camera module. The total length of the device is 110 mm, and the diameter is 11 mm, and it can be inserted into a 12 mm trocar. For the integrated light source, an LED array fits around the camera module, and provides lighting with low power requirements.

This insertable instrument has only a single camera, but an alternative insertable instrument includes 2 cameras side by side in a single camera module, thus sharing the pan/tilt axes but providing stereo 3D imaging. Including 2 cameras in a side-by-side design increases the device's diameter to 15 mm. Modular design was used advantageously to make the device components interchangeable and extendable.

After the surgeon anchors a surgical device comprising the insertable instrument onto the abdomen wall, he can use the Joystick to position the camera to the desired surgical viewpoint using the Pan and Tilt motions. The intensity of illumination can be adjusted manually through the control panel. FIG. 11 is a diagram of the prototype built, and the configuration of the open-look control system is shown in FIG. 12. The solid-line blocks show the current system's functions, which include Joystick control, video display, pan/tilt motion control, and LED light source control. The dot-line blocks show additional functions such as 3D display, voice control, and surgical tools. The computer used is a standard PC (Intel Pentium III, 863 MHz, 384 MB RAM) with a Hauppauge frame grabber and a motion control board. The camera system is a single-board CCD videocamera (KS600, NET USA, Inc.).

This system also can digitally control the light intensity of the LED light source, and may comprise the PC and a 2 meter wire bundle containing control and imaging cables that connects directly to the device.

The motion control board is a National Instruments NIDAQ PCI-6713 board with a SCB 68 break-out board, which can control the motor's direction, position and velocity. The pan and tilt motors use brushless DC motors. The NIDAQ board generates a series of control square waves to motor drivers (BLCPS.0002.8, Smoovy, Inc.), which directly output appropriate sequence current to the motor coils to drive the motor at certain speeds.

### Example IV

### Lens and Camera Design

A standard endoscope uses a series of relay lenses to transmit the image to the CCD camera sensor outside of the body. This approach protects the fragile electronics from the body fluid and moisture. The user may dispose of low-cost components, such as the lens and mechanical components, while saving the expensive parts such as CCD camera head and motors after surgical procedures. In the devices of the present invention, sealing can both protect the components and enable their recycling for future use.

The optical characteristics of the lens were determined by starting with data from a standard laparoscope which has a view angle of about 50 degrees. For Example III above, a ¼ inch color video CCD camera head was used; this camera head had an outside diameter of 6.5 mm (NET USA Inc, CSH-1.4-V4-END-R1). The camera has active pixels of 752(H)×582(V) at PAL system, which can provide 450 TV lines in horizontal resolution and 420 TV lines in vertical resolution. The CCD sensor has an active area of circle 4.5 mm in diameter.

Despite evidence that patients benefit from the laparoscopic approach, at this time very few complex abdominal surgeries are performed laparoscopically. These include colectomy, bariatric cases, abdominal hysterectomy, and solid organ resections (for example, pancreatectomy, hepatectomy, and splenectomy). One reason that such surgeries are rarely performed is that current devices and procedures require the presence of an experienced camera operator to assist the surgeons. Because many surgeons currently do not have such assistance, they can benefit from the potential ability to perform more complex cases laparoscopically by having a reliable, easily controlled image to view during the procedures. This applies to all laparoscopic cases, including complex cases such as colectomy, bariatric cases, and solid organ removal. Thus, the devices and methods of the present invention may enable more surgeons to learn and complete surgeries laparoscopically by providing an intuitively controlled image source, thus making the procedure easier to execute without additional help to the surgeon.

It should be noted that the Examples above are merely illustrative, not limiting to the present invention, and that additional embodiments and variations are possible without departing from the spirit of the invention.

What is claimed is:

1. A surgical device comprising:
(a) an insertable instrument; and
(b) a trocar attached to the insertable instrument.

2. The surgical device of claim 1, wherein the trocar is attached to the insertable instrument by adhesion, a hook, a magnet, a clamp, a snap, welding, a thread or string, a wire, soldering, a pin, a screw, threading or a plastic connector.

3. The surgical device of claim 1, wherein the insertable instrument is chosen from a cutting implement, a dissector, a retractor, a cauterizer, a suction device, a probe, a magnifier, scissors, tweezers, forceps, a coagulator, an ultrasonically activated shears or probe, other monopolar or bipolar cutting and coagulating devices, any other energy source for cutting, dissection, or hemostasis.

4. The surgical device of claim 1, wherein the insertable instrument is an imaging device.

5. The surgical device of claim 4, wherein the imaging device comprises a camera.

6. The surgical device of claim 5, wherein the imaging device comprises a scope.

7. The surgical device of claim 6, wherein the scope is chosen from an endoscope, a laparoscope, a thoracoscope, a gastroscope or a colonoscope.

8. The surgical device of claim 7, wherein the scope is rigid or flexible.

9. The surgical device of claim 1, wherein at least a portion of the surgical device is attached to the wall of the abdomen of a patient by adhesion, a hook, a magnet, a clamp, a snap, welding, a thread or string, a wire, soldering, a pin, a screw, threading or a plastic connector.

10. The surgical device of claim 6, wherein the scope provides direct image transmission or fiberoptic image transmission.

11. The surgical device of claim 6, wherein the scope is a videoscope with a video camera in it.

12. The surgical device of claim 3, wherein the insertable instrument is a probe chosen from an ultrasound probe or a radioactivity detection probe.

13. The surgical device of claim 4, wherein the surgical device is a microscopic or spectroscopic imaging device.

14. The surgical device of claim 4, wherein the imaging device is movable on one or more of the following: a pan axis, a tilt axis, a translation axis and a zoom axis.

15. The surgical device of claim 1, further comprising an actuation mechanism.

16. The surgical device of claim 15, wherein the actuation mechanism is mechanically or electrically controlled.

17. The surgical device of claim 16, wherein the actuation is motor driven, remotely controlled or directly controlled.

18. A surgical device comprising an insertable instrument attached to a trocar to provide an insertable instrument/trocar combination,
wherein the insertable instrument is capable of being inserted into a single incision in the skin of a patient to permit a surgical procedure on the patient.

19. The surgical device of claim 18, wherein the insertable instrument comprises an imaging device.

20. The surgical device of claim 18, wherein the surgical device is capable of being inserted into a single incision to create an opening having a diameter of about 3 cm or less in the skin of the patient.

21. The surgical device of claim 19, wherein the surgical device is capable of being inserted into a single incision to create an opening having a diameter of about 1 cm to about 3 cm in the skin of a patient.

22. The surgical device of claim 19, wherein the surgical device is capable of being inserted into a single incision to create an opening having a diameter of about 5 mm to about 10 mm in the skin of a patient.

23. The surgical device of claim 18, wherein the insertable instrument attached to the trocar by a magnet.

24. A method of performing a procedure on a patient, the method comprising the steps of:
(a) making an incision in the skin of a patient to provide an opening having a diameter of about 3 cm or less; and
(b) inserting an insertable instrument having a trocar attached thereto through the opening.

25. A method of performing a surgical procedure on a patient, the method comprising:
(a) inserting a trocar into an incision on the surface of a patient's body, such that the trocar touches the incision and maintains an opening in the skin of the patient;
(b) inserting an insertable instrument into the interior of a patient's body through the trocar; and
(c) attaching the trocar to the insertable instrument via a point of attachment on the surface of the trocar or the insertable instrument; wherein at least a portion of the point of attachment is within the interior of the patient's body.

26. A method of performing a surgical procedure on a patient, the method comprising:
(a) making an incision in the skin of a patient to provide an opening having a diameter of about 3 cm or less;
(b) attaching a trocar to an insertable instrument to form an insertable instrument/trocar combination; and
(c) inserting at least a portion of the insertable instrument/trocar combination through the incision into the patient's body, such that the trocar touches the incision and maintains an opening in the skin of the patient, and such that at least a portion of the insertable instrument is within the interior of the patient's body.

27. The method of claim 25 or 26, further comprising the step of inserting at least one additional item through the opening, the additional item chosen from a grasper, a dissector, a grasper, scissors, a scalpel, a tool delivering cautery, ultrasonic energy or a laser, a clip applier, a stapler, a probe, a suction device, a delivery device for an adhesive, anti-adhesive or hemostatic agent, a mesh, a prosthesis or an implantable device.
